(19) United States
(12) Patent Application Publication
(10) Pub. No.: US 2019/0352727 A1
(43) Pub. Date: Nov. 21, 2019

(54) METHOD FOR ANALYZING SOMATIC CELL REPROGRAMMING, AND METHOD FOR SETTING UP QUALITY EVALUATION CRITERIA FOR iPS CELL USING THE METHOD

(71) Applicant: Olympus Corporation, Tokyo (JP)

(72) Inventor: Ryutaro Akiyoshi, Tokyo (JP)

(73) Assignee: Olympus Corporation, Tokyo (JP)

(21) Appl. No.: 16/297,612

(22) Filed: Mar. 9, 2019

Related U.S. Application Data
(63) Continuation of application No. PCT/JP2016/084511, filed on Nov. 21, 2016.

(51) Int. Cl. C12Q 1/6897; C12N 5/074; G01N 33/50; G01N 33/52; G16B 40/10

(52) U.S. Cl. CPC C12Q 1/6897; C12N 5/0696; G16B 40/10; G01N 33/52; G01N 33/5023

(57) ABSTRACT

The disclosed technology provides a method for analyzing a somatic cell reprogramming, the method including acquiring luminescence images associated with luminescences attributed to expression of luminescent reporter proteins during a period of culturing cells transfected with a nucleic acid encoding plural types of transcription factors required for the somatic cell reprogramming and a nucleic acid encoding the luminescent reporter proteins configured to be co-expressed with at least one of the plural types of transcription factors in S12, quantifying luminescence intensities of the luminescences based on the luminescence images in S13, and evaluating expression states of the transcription factors to be co-expressed with the luminescent reporter proteins based on the luminescence intensities in S14, in which the expression states are indicators for evaluating whether a pluripotency has been acquired in the cell reprogramming process.

### CROSS-REFERENCE TO RELATED APPLICATION

This application is a continuation application of PCT Application No. PCT/JP2016/084511 filed on Nov. 21, 2016, which is hereby incorporated by reference in its entirety.

### TECHNICAL FIELD

The disclosed technology relates to a method for analyzing somatic cell reprogramming, and a method for setting up quality evaluation criteria for iPS cells using the method.

### DESCRIPTION OF THE RELATED ART

The iPS (induced pluripotent stem) cells are established through reprogramming by introducing initializing factors (e.g., Oct4, Klf4, Sox2, c-myc, Lin28, or L-myc) into somatic cells. It is known that the establishment efficiency of the iPS cells is low, and among the cells transfected with plural types of transcription factors necessary for initialization, a percentage of pluripotent iPS cells, that is, cells to develop into high-quality iPS cells is not higher than 1%.

National Publication of International Patent Application No. 2010-537634 describes a method of visually evaluating a colony morphology as a method for evaluating high-quality iPS cells. This evaluation is carried out after sufficient growth of the colony, i.e., three to four weeks after the onset of reprogramming induction. Cells evaluated as having good colony morphology are maintained thereafter, but among them, a percentage of actually-pluripotent iPS cells is 20 to 40%. For establishing iPS cells, at least 15 to 30 colonies should be continuously maintained in practice, considering that it is necessary to obtain at least three strains from one donor. As described hereinbefore, in the conventional quality evaluating method, it is difficult to efficiently select the iPS cells, resulting in high cost and great labor.

In “Eirini P. Papapetrou et al., Proc. Natl. Acad. Sci. USA. 2009 August; 106 (31): 12756-64. Stoichiometric and temporal requirements of Oct4, Sox2, Klf4, and c-Myc expression for efficient human iPS induction and differentiation,” initializing factors labeled with a fluorescent protein are expressed in cells, and the expression levels of the factors are analyzed. However, since this method detects fluorescent signals by irradiating the cells with an excitation light, the cells suffer various damages due to phototoxicity especially in a case of long-term observation. If the cells are damaged due to phototoxicity during reprogramming situated on the most upstream step in the iPS cell preparation process, the subsequent steps may be greatly affected.

In addition, Eirini P. Papapetrou et al. have been reported that when a vector containing an initializing factor Oct4 in a larger number than vectors containing other initializing factors Sox2, Klf4, and c-Myc is introduced into a cell to express the Oct4 in a larger number than the other initializing factors, the reprogramming efficiency is improved.

A method for analyzing a gene expression with small damage to cells is exemplified by a method for analyzing luminescent signals attributed to expression of a luminescent reporter gene. Since the expression analysis using the luminescent signals requires no excitation light, there is no influence of phototoxicity, autofluorescence, etc., and a gene expression analysis excellent in quantitativity is possible.

Japanese Patent Laid-Open No. 2014-176364 describes a method for evaluating a state of a stem cell by luminescence. This document describes a method in which a differentiation state at a stage of differentiation-inducing an iPS cell to various organs is evaluated by a promoter assay targeting a differentiation marker gene or an undifferentiation marker gene for the cell. In the promoter assay, a luminescent quantity reflecting the number of intrinsic transcription factors is quantified.

Conventionally, the timing of evaluating the colony morphology is after the colonies have sufficiently grown, and evaluation for the morphologically-matured iPS cells is emphasized. Thus, there is a demand for a method for analyzing expression levels of transcription factors at an initial stage of reprogramming, with small damage to the cells and high quantitativity.

In addition to the method for visually evaluating the quality of the colony morphology, a method for setting up evaluation criteria for selecting high-quality iPS cells is required.

### BRIEF SUMMARY OF EMBODIMENTS

An object of the disclosed technology is to provide a minimally invasive and highly quantitative method for analyzing somatic cell reprogramming, and a method for setting up quality evaluation criteria for iPS cells using the method.

One aspect of the disclosed technology provides a method for analyzing a somatic cell reprogramming, the method including acquiring luminescence images associated with luminescences attributed to expression of luminescent reporter proteins during a period of culturing cells transfected with a nucleic acid encoding plural types of transcription factors required for the somatic cell reprogramming and a nucleic acid encoding the luminescent reporter proteins configured to be co-expressed with at least one of the plural types of transcription factors, quantifying luminescence intensities of the luminescences based on the luminescence images, and evaluating the expression states of the transcription factors to be co-expressed with the luminescent reporter proteins based on the luminescence intensities, in which the expression states are indicators for evaluating whether the pluripotency has been acquired in the cell reprogramming process.

One aspect of the disclosed technology provides a method for setting up quality evaluation criteria for iPS cells using the analyzing method described hereinbefore, the setting-up method including

obtaining results of evaluating the expression states of the transcription factors,

evaluating qualities of iPS cells prepared by reprogramming the cells to obtain results of evaluating the qualities of the iPS cells, and

setting up the quality evaluation criteria for the iPS cells based on the relationship between the results of evaluating the expression states of the transcription factors and the results of evaluating the qualities of the iPS cells.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

### DETAILED DESCRIPTION OF THE EMBODIMENTS

In the following description, various embodiments of the technology will be described. For purposes of explanation, specific configurations and details are set forth in order to provide a thorough understanding of the embodiments. However, it will also be apparent to one skilled in the art that the technology disclosed herein may be practiced without the specific details. Furthermore, well-known features may be omitted or simplified in order not to obscure the embodiment being described.

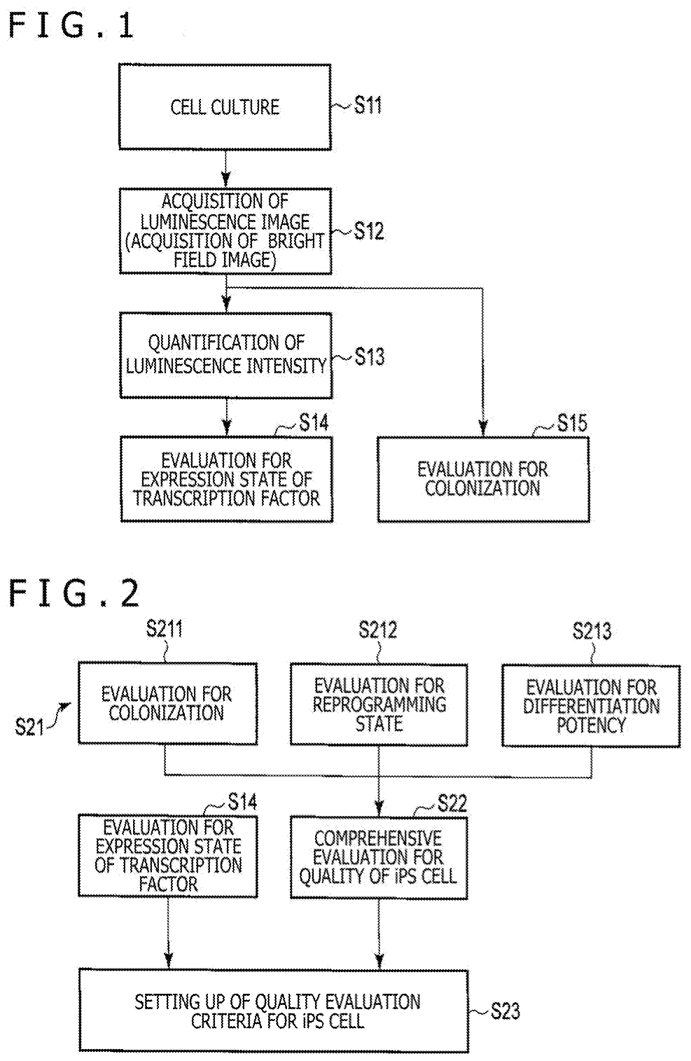

FIG. 1 is a flow chart of a main treatment of a method for analyzing a somatic cell reprogramming according to an embodiment of the disclosed technology.

FIG. 2 is a flow chart of a main treatment of a method for setting up quality evaluation criteria for iPS cells according to an embodiment of the disclosed technology.

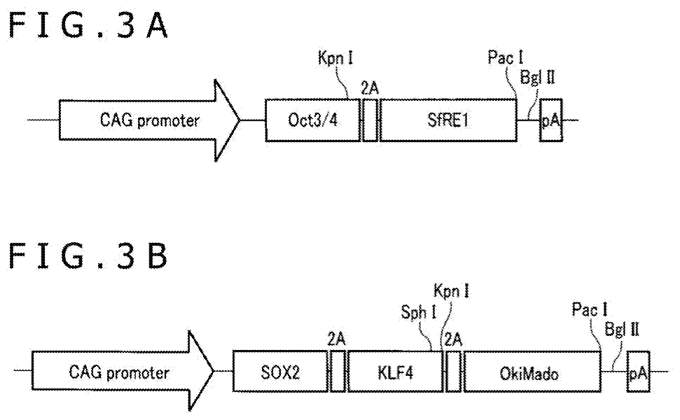

FIG. 3A is a schematic diagram illustrating a configuration of a pCXLE-hOCT3/4-SfRE1 vector used in Examples.

FIG. 3B is a schematic diagram illustrating a configuration of a pCXLE-hSK-OkiMado vector used in Examples.

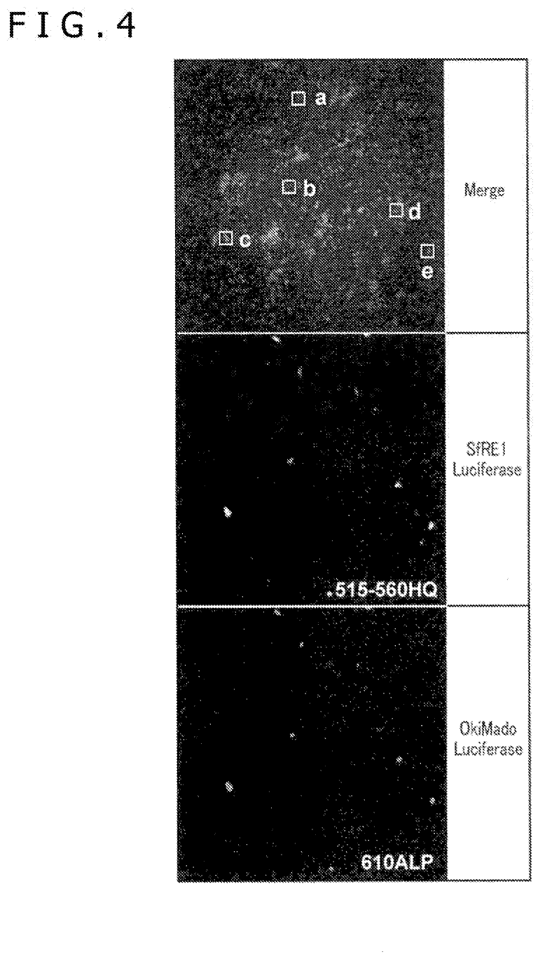

FIG. 4 depicts images indicating a luminescence image of luminescence attributed to an SfRE1 luciferase, a luminescence image of luminescence attributed to an OkiMado luciferase, and an image (Merge) obtained by superposing the luminescence images and a phase difference observation image, for cells on the first day after introduction of the vectors.

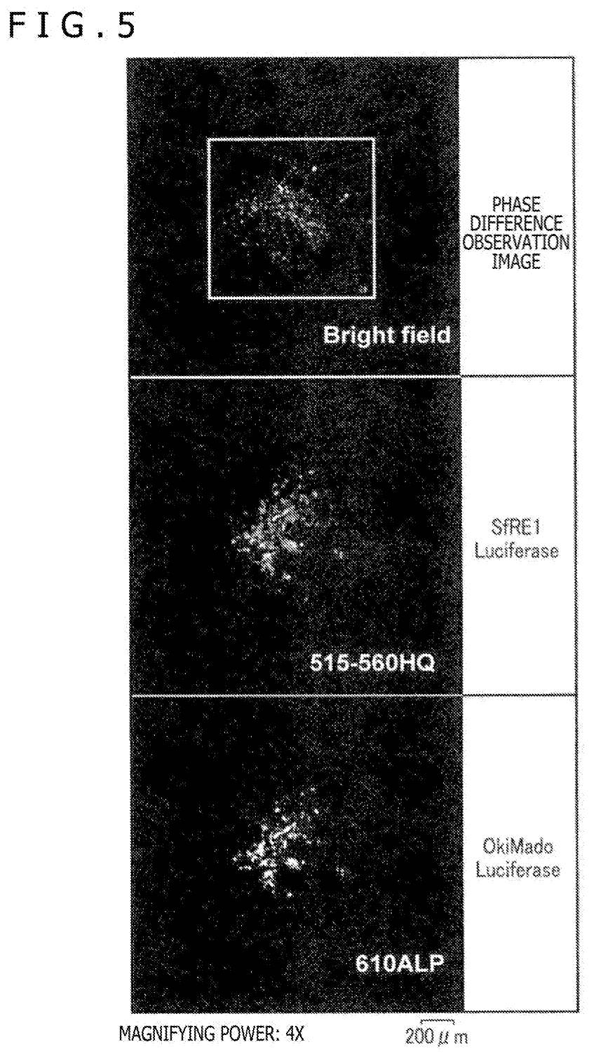

FIG. 5 depicts images indicating a luminescence image of luminescence attributed to the SfRE1 luciferase, a luminescence image of luminescence attributed to the OkiMado luciferase, and a phase difference observation image, for iPS-like cell colonies on the eighth day after introduction of the vectors.

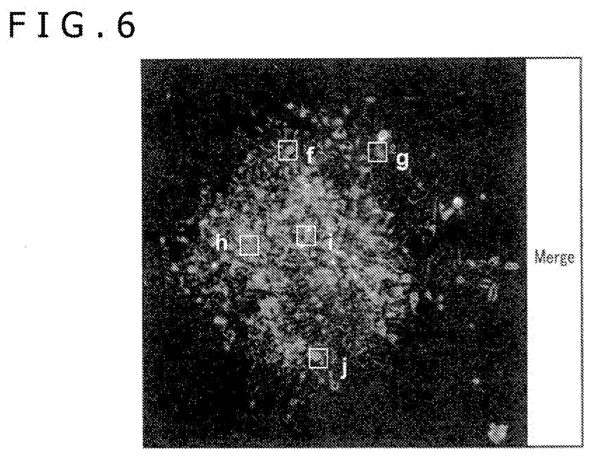

FIG. 6 depicts an image (Merge) obtained by enlarging a range indicated in FIG. 5 and superposing the luminescence image and the phase difference observation image.

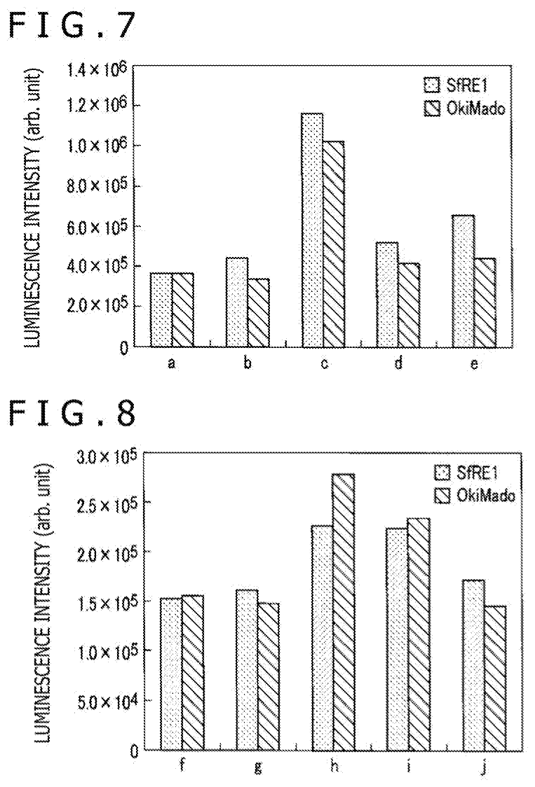

FIG. 7 is a graph illustrating a luminescence intensity attributed to expression of the SfRE1 luciferase and a luminescence intensity attributed to expression of the OkiMado luciferase, in each cell selected from the luminescence images illustrated in FIG. 4.

FIG. 8 is a graph illustrating the luminescence intensity attributed to expression of the SfRE1 luciferase and the luminescence intensity attributed to expression of the OkiMado luciferase, on five sites in a colony selected from the luminescence image illustrated in FIG. 6.

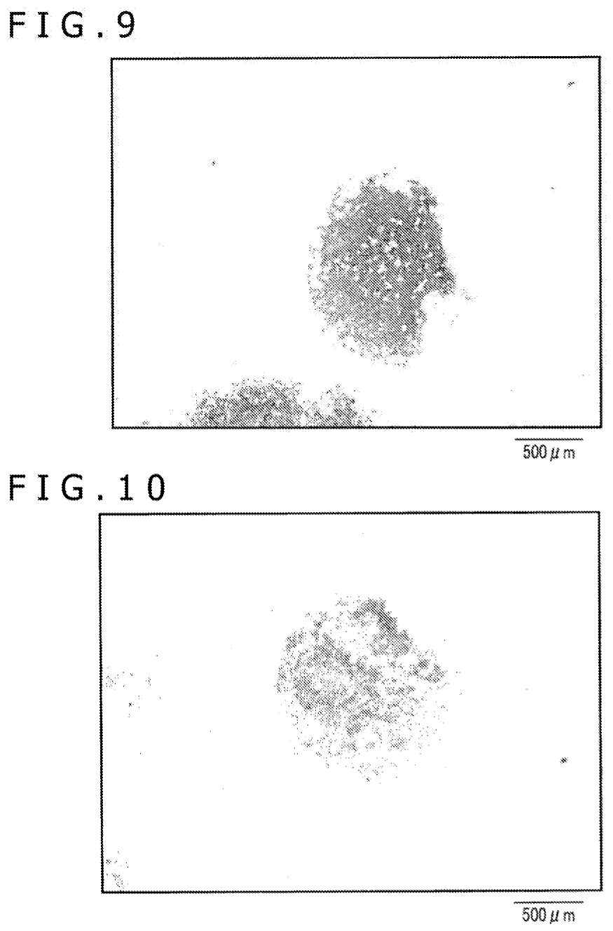

FIG. 9 depicts a bright field image of the iPS-like cell colony stained with alkaline phosphatase eight days after introduction of a modified vector.

FIG. 10 depicts a bright-field image of the iPS-like cell colony stained with the alkaline phosphatase eight days after introduction of a control vector.

### DESCRIPTION OF EMBODIMENT

Hereinafter, the disclosed technology will be described in detail, but the following description is intended to describe the disclosed technology and not intended to restrict the disclosed technology.

### Method for Analyzing Somatic Cell Reprogramming

FIG. 1 is a flow chart of a main treatment of a method for analyzing a somatic cell reprogramming according to an embodiment of the disclosed technology. A method according to an embodiment of the disclosed technology includes culturing cells in S11, obtaining luminescence images in S12, quantifying luminescence intensities in S13, evaluating expression states of transcription factors in S14, and evaluating colonization in S15. The “reprogramming” refers to a phenomenon that a differentiated cell is transformed into a pluripotent stem cell.

### Cell Culture

Cells transfected with a nucleic acid encoding plural types of transcription factors required for the somatic cell reprogramming and a nucleic acid encoding luminescent reporter proteins configured to be co-expressed with at least one of the plural types of transcription factors are cultured in S11. In the following description, the former nucleic acid is also referred to as a “transcription factor-encoding nucleic acid,” and the latter nucleic acid is also referred to as a “luminescent reporter protein-encoding nucleic acid.” In the present specification, the nucleic acid is synonymous with gene, and for example, is DNA.

The original cells to be transfected with the “transcription factor-encoding nucleic acid” and the “luminescent reporter protein-encoding nucleic acid” are somatic cells, and the somatic cells transfected with these nucleic acids can be transformed into iPS cells during the culture period. The cells can be cultured in accordance with a known method. Preferably, the cells are cultured on a microscope except for times of operations such as medium replacement. As the somatic cells, for example, human peripheral blood mononuclear cells (hereinafter referred to as PBMC) can be used, but the somatic cells are not limited thereto. The cells are continuously cultured over a period of the method for analyzing the somatic cell reprogramming.

In the somatic cells transfected with the “transcription factor-encoding nucleic acid” and the “luminescent reporter protein-encoding nucleic acid,” the somatic cell reprogramming is induced by expressing the “transcription factor-encoding nucleic acid” during the culture period.

Preferably, the “transcription factor-encoding nucleic acid” and the “luminescent reporter protein-encoding nucleic acid” are introduced so as to be persistently expressed without being incorporated into host chromosomes. For example, the “transcription factor-encoding nucleic acid” and the “luminescent reporter protein-encoding nucleic acid” are preferably introduced into the somatic cells in such a way that the nucleic acids are in a form of episomal vectors. In this case, for the episomal vector, a commercially available product may be used, or a modified vector prepared by incorporating the “luminescent reporter protein-encoding nucleic acid” into the commercially available episomal vector may be used. As the commercially available episomal vector, for example, pCXLE-hOCT3/4-shp 53-F (Addgene), pCXLE-hSK (Addgene), and pCXLE-hUL (Addgene) can be used.

“Nucleic acids encoding plural types of transcription factors” may be incorporated into individual vectors, or into one vector. When the “nucleic acids encoding plural types of transcription factors” are incorporated into one vector, for example, it is preferable to insert the nucleic acids by polycistronically linking the nucleic acids via a 2A sequence, an IRES sequence, or the like in a foot-and-mouth disease virus. As an example, plural types of vectors, each one of which is transfected with a nucleic acid encoding one type of transcription factor or two types of transcription factors incorporated via the 2A sequence, are prepared. The vectors are mixed, and the nucleic acids encoding all transcription factors required for inducing the iPS cells are introduced into the somatic cells. The gene transfer manipulation can be carried out in accordance with a known method.

In addition to the “transcription factor-encoding nucleic acid” and the “luminescent reporter protein-encoding nucleic acid,” a “nucleic acid encoding an additional factor for increasing the reprogramming efficiency” may be introduced into the somatic cells. As the “additional factor for increasing the reprogramming efficiency,” a factor known to increase the reprogramming efficiency, for example, a dominant negative mutation-introduced mouse p53 and EBN A1 can be used. Preferably, the “additional factor for increasing the reprogramming efficiency” in a form of an episomal vector is introduced into the somatic cells. In this case, a commercially available episomal vector, for example, pCE-mp 53DD (Addgene) and pCXB-EBNA1 (Addgene) can be used.

As the “plural types of transcription factors required for the somatic cell reprogramming,” a combination of transcription factors known to induce the somatic cell reprogramming, for example, a combinations of Oct3/4, Klf4, Sox2, c-myc, Lin28, and L-myc, a combination of Oct3/4, Klf4, Sox2, Lin28, and L-myc, a combination of Oct3/4, Klf4, Sox2, and c-myc, or a combination of Oct3/4, Klf4, and Sox2 can be used. Number of types of the transcription factors required for the somatic cell reprogramming is, for example, three to six.

As an example, the “transcription factor-encoding nucleic acid” can be introduced into the somatic cells by using a vector set composed of a first vector including an Oct3/4-encoding nucleic acid, a second vector including a Sox2-encoding nucleic acid and a Klf4-encoding nucleic acid, and a third vector including an L-myc-encoding nucleic acid and a Lin28-encoding nucleic acid. Herein, at least one of the first vector, the second vector, and the third vector includes the “luminescent reporter protein-encoding nucleic acid” at a position where the nucleic acid can be co-expressed with the “transcription factor-encoding nucleic acid” included in the vector. In addition, when the “transcription factor-encoding nucleic acid” is introduced into somatic cells using the vector set composed of the first to third vectors described hereinbefore, a fourth vector including a nucleic acid encoding the dominant negative mutation-introduced mouse p53, and a fifth vector including a nucleic acid encoding the dominant negative mutation-introduced mouse EBN A1 may be incorporated into the vector set.

The “luminescent reporter protein-encoding nucleic acid” is configured to be co-expressed with at least one of the plural types of transcription factors. For example, preferably, the “luminescent reporter protein-encoding nucleic acid” is configured to polycistronically link to the “transcription factor-encoding nucleic acid” via the 2A sequence, the IRES sequence, or the like in the foot-and-mouth disease virus. For example, preferably, the “luminescent reporter protein-encoding nucleic acid” is configured to polycistronically link to the “transcription factor-encoding nucleic acid” via the 2A sequence. The position of the “luminescent reporter protein-encoding nucleic acid” may be any position where both the transcription factor and the luminescent reporter protein can be expressed, and may be located upstream of the “transcription factor-encoding nucleic acid” or downstream thereof.

In the present specification, the “luminescent reporter protein” is used in contrast to a fluorescent protein such as a green fluorescent protein (GFP), and functions so as to emit light without irradiation with excitation light. The “luminescent reporter protein” is preferably a bioluminescent protein, for example, luciferase. Since bioluminescence is not caused in dead cells that have lost biological reactions, the bioluminescence is preferable from the viewpoint that living cells excluding cells inactivated due to apoptosis or the like during the culture period can be selectively evaluated.

The “luminescent reporter protein” may be configured to be co-expressed with one of plural types of transcription factors, or to be co-expressed with all of plural types of transcription factors. The transcription factor configured to be co-expressed with the luminescent reporter protein is at least one selected from a group including Oct3/4, Klf4, Sox2, c-myc, Lin28 and L-myc.

When the “luminescent reporter protein” is configured to be co-expressed with two or more of the plural types of transcription factors, the expression states of the two or more transcription factors can be evaluated. Thereby, the somatic cell reprogramming can be more accurately analyzed.

When there are two or more types (e.g., two to six types) of transcription factors configured to be co-expressed with the luminescent reporter proteins, each of the luminescent reporter proteins configured to be co-expressed with the two or more types of transcription factors has a luminescence property allowing the luminescent reporter proteins to be detected distinguishably with any of the other luminescent reporter proteins. That is, two or more types of luminescent reporter proteins have the luminescence property allowing the luminescent reporter proteins to be detected distinguishably with each other. Herein, the luminescence property is, for example, an emission wavelength. As described hereinbefore, the two or more types of luminescent reporter proteins have luminescence property allowing the luminescent reporter proteins to be detected distinguishably with each other, and thus, in the luminescence image obtained in the subsequent step, information of the luminescence attributed to each expression of the luminescent reporter proteins is also distinguished from information of the luminescence attributed to expression of any other luminescent reporter proteins. Furthermore, in the subsequent quantification step, the luminescence intensity of the luminescence attributed to each expression of the luminescent reporter proteins is individually (i.e., for each type of the luminescent reporter proteins) quantified based on these luminescence images.

As the two or more types of luminescent reporter proteins having the luminescence property allowing the luminescent reporter proteins to be detected distinguishably with each other, commercially available products such as an Eluc luciferase causing green luminescence, a CRB luciferase causing red luminescence, and a Renilla luciferase causing blue luminescence can be used.

In addition, the luciferase used as the luminescent reporter protein may be a modified luciferase which is modified so as to have a high luminescence intensity. As the modified luciferase, for example, an SfRE1 luciferase (SEQ ID NO: 1), an OkiMado luciferase (SEQ ID NO: 2), or the like can be used. Note that the combination of the transcription factor and the luciferase is not particularly limited.

Since the luminescent reporter protein is co-expressed with the transcription factor, the expression timing and the expression level of the luminescent reporter protein-encoding gene can be regarded as corresponding to those of the transcription factor. When using the luciferase as the luminescent reporter protein, a luciferase protein produced in the cell by expression of the luciferase gene catalyzes a luciferin provided to the cell, resulting in luciferin luminescence. Consequently, the expression state and the like of the transcription factor can be estimated based on the luminescence.

### Acquisition of Luminescence Image

The luminescence attributed to the expression of the luminescent reporter protein is imaged to acquire a luminescence image in S12.

The luminescence image is acquired during the culture treatment period in S11 (culture period), preferably at the initial stage of the reprogramming. Herein, the initial stage of the reprogramming may include a prestage of colonization, where cells are still primarily at a two-dimensional proliferative phase. In addition, the initial stage of the reprogramming may include an initiation stage of colonization, where cells start to three-dimensionally upheave by clusterization. The luminescence image is acquired, for example, before completion of the reprogramming. The luminescence image is acquired at least once, at an earlier stage than the conventional timing for evaluating the colony morphology, specifically within three weeks after introduction of the “transcription factor-encoding nucleic acid” (i.e., after the onset of reprogramming induction). More specifically, the luminescence image is acquired at least once, within three weeks after the day of introduction of the “transcription factor-encoding nucleic acid.”

In addition, the luminescence image is acquired at least once, preferably before colonization, specifically within one week after introduction of the “transcription factor-encoding nucleic acid.” More specifically, the luminescence image is acquired at least once, within one week after the day of introduction of the “transcription factor-encoding nucleic acid.”

Preferably, the luminescence images are repeatedly acquired over time. The imaging is continuously carried out at an arbitrary interval. The imaging is generally carried out at an interval of 10 to 30 minutes, for example, an interval of 10 minutes. In addition, the period for one imaging is arbitrarily set. Although an exposure time of a camera for one imaging is set to, for example, three to five minutes, the exposure time can be appropriately adjusted such that sufficient luminescent signals can be detected. The interval between the images is set to an arbitrary time interval which is longer than the exposure time for generating the image allowing the luminescence image to be analyzed by imaging elements. On the other hand, the reprogramming process is carried out by the week, and thus, even if each one image is taken every other day or every other week, the cells before maturation to iPS cells can be effectively evaluated.

The period allowing acquisition of the luminescence image is limited to periods for which the “transcription factor-encoding nucleic acid” and the “luminescent reporter protein-encoding nucleic acid” can be maintained in the cells. When the “transcription factor-encoding nucleic acid” and the “luminescent reporter protein-encoding nucleic acid” in a form of episomal vectors are introduced into the cells, the luminescence can be continuously imaged from immediately after the onset of reprogramming induction, that is, from introduction of the episomal vector, until the release of the episomal vectors outside of the cells.

The luminescence image is preferably acquired in a light-shielded environment. More specifically, the luminescence image is preferably acquired in accordance with a method with small external influence in a light-shielded environment for imaging bioluminescence. The luminescence image can be acquired optionally using a filter in a light-shielded environment. In the case of detecting only one type of luminescent reporter protein, the luminescence image may be acquired with using no filter in a light-shielded environment. In the case of detecting plural types of luminescent reporter proteins, the luminescence image is preferably acquired after spectral separation optionally using a filter in a light-shielded environment. Also, the luminescence image can be acquired as an image in which luminescences attributed to expression of the plural types of luminescent reporter proteins are superposed, without spectral separation through a filter. Also, the luminescence image can be acquired as a color image using a color CCD camera or a color CMOS camera.

The luminescence image can be acquired by using a luminescence imaging apparatus, for example, a luminescence imaging apparatus including a filter mainly for allowing passage of light having a specific wavelength corresponding to the luminescence, an imaging element converting the light which has passed through the filter into electric signals, and a processing portion creating the luminescence image from the electric signals. As the luminescence imaging apparatus, a luminescence imaging system LV200 (Olympus Corporation) having both a culturing function and an imaging function can be used. The luminescence images can be acquired in the whole process of reprogramming by performing the imaging function at a desired timing while performing the culturing function. Thus, after the luminescence images are time-serially acquired in the whole process, an image suitable for analysis can be selected from the plurality of luminescence images including luminescence images taken at the initial stage of reprogramming. Furthermore, in the plurality of colonies which have been started to be cultured at the same time in the same culture chamber, luminescence images with different elapsed times are selected, so that colonies having the same growth degree can be evaluated among colonies having different growth rates.

Preferably, a bright field image is acquired together with the luminescence image. More preferably, the bright field image is acquired at almost the same timing as acquisition of the luminescence image. The bright field image refers to an image acquired using illumination light not based on luminescence, which allows the positions, morphologies, and the like of cells or colonies to be observed. The bright field image includes a phase difference observation image and a differential interference contrast (DIC) observation image. The bright field image may be acquired at almost the same timing as acquisition of the luminescence image, or may be acquired at any timing independently of acquisition of the luminescence image. In an automated system like the luminescence imaging system (LV200), the imaging functions of both the luminescence image and the bright field image can also be performed while switching the functions at a predetermined timing under a sufficient light-shielded condition.

### Quantification of Luminescence Intensity

Based on the acquired luminescence image, the luminescence intensity of the luminescence attributed to the expression of the luminescent reporter protein is quantified in S13.

In the case in which luminescent reporter protein to be detected is one type, the luminescence intensity detected at a wavelength range of luminescence of the luminescent reporter protein can be directly quantified.

In the case in which luminescent reporter proteins to be detected are two or more types, preferably, light is first spectrally separated by a filter to acquire a luminescence image for each predetermined wavelength range. Subsequently, preferably, the acquired luminescence image is unmixed to exclude portions of overlapped emission wavelength ranges of the plural types of luminescent reporter proteins, and then, each of the luminescence intensities of the luminescences attributed to expression of plural types of luminescent reporter proteins can be sorted and quantified. In the subsequent evaluation step, a ratio of the luminescence intensities can also be calculated from the quantitative results of the luminescence intensities attributed to the expression of the respective luminescent reporter proteins acquired hereinbefore.

The luminescence intensity may be quantified as a total value of the luminescence intensities of the luminescences attributed to expression of plural types of luminescent reporter proteins. In this case, information of the whole expression level of plural types of luminescent reporter proteins can be obtained.

In addition, in the case in which luminescent reporter proteins to be detected are two or more types, the luminescence intensities can also be quantified by color-separating the luminescences attributed to expression of plural types of luminescent reporter proteins based on a color image and then sorting information of each luminescence.

In a case in which acquisition of the luminescence images over time is repeated, quantification of the luminescence intensities is preferably repeated over time. Also in this case, a ratio of the luminescence intensities can be calculated at each time point for quantifying the luminescence intensity, in the subsequent evaluation step. The quantification of the luminescence intensities and the calculation of the ratio of the luminescence intensities may be automated.

### <<Evaluation for Expression State of Transcription Factor>>

Based on the quantified luminescence intensity, the expression state of the transcription factor configured to be co-expressed with the luminescent reporter protein is evaluated in S14. The evaluation for the expression state of the transcription factor preferably includes determining the expression level of the transcription factor from the quantitative result of the luminescence intensity.

In a case in which luminescent reporter protein used is one type, the luminescence intensity attributed to the expression of the luminescent reporter protein can be directly converted into the expression level of the transcription factor configured to be co-expressed with the luminescent reporter protein.

In a case in which luminescent reporter proteins used are plural types, the evaluation for the expression state of the transcription factor may include determining the expression level of the transcription factor from the luminescence intensity quantified individually (i.e., for each type of the luminescent reporter proteins). In this case, preferably the relationship between the expression level of the specific transcription factor and each luminescence intensity of plural types of luciferases is determined in advance such that the expression level of the transcription factor can be corrected even if the luminescent reporter protein for coexpression is changed.

In a case in which the luminescence intensities of the luminescences attributed to the expression of plural types of luminescent reporter proteins are quantified as a total value thereof, the expression levels of the transcription factors are similarly quantified as a whole expression level of the plural types of transcription factors.

The expression state of the transcription factor is evaluated preferably for cells at the initial stage of the reprogramming, more preferably for cells within three weeks after introduction of the “transcription factor-encoding nucleic acid,” even more preferably for cells within one week after introduction of the “transcription factor-encoding nucleic acid.” The expression state of the transcription factor may be evaluated immediately after acquiring the luminescence image, or may be evaluated using the previously-acquired luminescence image.

Furthermore, in a case in which luminescent reporter proteins used are plural types, the evaluation for the expression state of the transcription factor may include determining a ratio of the luminescence intensities from the luminescence intensities quantified individually (i.e., for each type of the luminescent reporter proteins).

The expression state of the transcription factor may be evaluated for the quantitative result of the luminescence intensity at onetime point. Alternatively, in a case in which the luminescence image is repeatedly acquired over time, the expression state of the transcription factor may be evaluated by evaluating the time-dependent change in the expression state of the transcription factor configured to be co-expressed with the luminescent reporter protein.

Thus, the expression state of the transcription factor can be evaluated by evaluating the time-dependent change in the expression intensity of the specific transcription factor associated with progress of the reprogramming and/or the time-dependent change in the ratio of the expression intensities of plural types of transcription factors. The expression state of the transcription factor is an indicator for evaluating acquisition of pluripotency in the cell reprogramming process. Herein, the “acquisition of pluripotency” means establishment of high-quality iPS cells. The high-quality cells can be sorted out at an early stage by evaluating acquisition of the pluripotency, and thus, the production efficiency of the iPS cells can be dramatically improved.

### Evaluation for Colonization

In addition to the evaluation for the expression state of the transcription factor in S14, the evaluation for colonization in S15 may be carried out. The evaluation for colonization in S15 can be carried out by visually confirming whether or not the colony is formed. In a case in which colonization is confirmed in the colonization evaluation in S15, this case indicates that the “transcription factor-encoding nucleic acid” is introduced into the cells and expressed. In addition, preferably the colonization evaluation in S15 is carried out from a bright field image. The bright field image is not particularly limited as long as the image allows colonization to be observed. The bright field image may be, for example, a phase difference observation image or a differential interference contrast (DIC) observation image.

The colonization can be evaluated on and after the seventh day after introduction of the “transcription factor-encoding nucleic acid” into the cells (i.e., after the onset of reprogramming induction).

### <<Method for Setting up Quality Evaluation Criteria for iPS Cell Using the Method for Analyzing Somatic Cell Reprogramming>>

FIG. 2 is a flow chart of a main treatment of a method for setting up quality evaluation criteria for iPS cells according to an embodiment of the disclosed technology. The method according to an embodiment of the disclosed technology includes, by using the above method for analyzing the somatic cell reprogramming, evaluating the expression state of the transcription factor in S14, evaluating the quality of the iPS cells in S21 and S22, and setting up the quality evaluation criteria for the iPS cells based on the relationship between the result of evaluating the expression state of the transcription factor and the result of evaluating the quality of the iPS cells in S23.

The evaluation for the expression state of the transcription factor in S14 can be carried out for cells within preferably three weeks after, more preferably one week after introduction of the “transcription factor-encoding nucleic acid” into the cells, as described hereinbefore. The Evaluation for the quality of the iPS cells in S21 and S22 can be carried out preferably three to four weeks after introduction of the “transcription factor-encoding nucleic acid” into the cells.

### Evaluation for Quality of iPS Cell

Colonies exhibiting various expression states of the transcription factor are selected from colonies subjected to analysis of the somatic cell reprogramming, followed by an evaluation conventionally used as the quality evaluation for iPS cells in S21, and based on the result, the quality of the iPS cells is comprehensively evaluated in S22.

In order to evaluate the quality of the iPS cells in S21 and S22, the colonies exhibiting various expression states (i.e., expression patterns) of the transcription factor are selected, and for example, colonies exhibiting expression patterns A, B, and C are selected. The “expression pattern” may be an expression pattern of one type or plural types of transcription factors at a specific time point, or a pattern of time-dependent change in the expression of one type or plural types of transcription factors. A plurality of (e.g., 10 to 100) colonies exhibiting specific expression patterns (e.g., expression patterns exhibiting transcription factor expression levels exceeding a predetermined threshold value) are selected, so that, in the subsequent step, the accuracy of the association between the result of evaluating the expression state of the transcription factor in S14 and the result of evaluating the quality of the iPS cells in S21 and S22 is improved, and therefore, the accuracy of the quality evaluation criteria set up for the iPS cells are also improved.

The quality of the iPS cells can be evaluated in accordance with the same method as known evaluation methods conventionally used for evaluating the quality of the iPS cells. For example, the evaluation for the quality of the iPS cells in S21 and S22 includes at least one selected from a group including evaluation for the colony morphology in S211, evaluation for the reprogramming state in S212, and evaluation for the differentiation potency in S213. The evaluation for the quality of the iPS cells preferably includes two or more selected from a group including evaluation for the colony morphology, evaluation for the reprogramming state, and evaluation for the differentiation potency, and more preferably, includes all of them. When a plurality of evaluations are carried out, the evaluation for the quality of the iPS cells includes comprehensive evaluation for a plurality of evaluation results. For example, in a case in which at least one evaluation result in the plurality of evaluations is poor, the quality of the iPS cells can be comprehensively evaluated as low.

### (Evaluation for Colony Morphology)

The colonization can be evaluated, for example, by at least one selected from a group including evaluation for the roundness of the colony, evaluation for the size of the colony, and evaluation for the clarity of the colony outline. The colony morphology may be evaluated by visual observation or by digitalization based on arbitrary determination criteria, and the digitalization process may be automated. Based on the colony morphology, a mature iPS cell can be screened, and cells exhibiting apparent abnormal morphology out of the mature iPS cells can be excluded.

### (Evaluation for Reprogramming State)

The reprogramming state can be evaluated, for example, by analyzing an alkaline phosphatase activity, a karyotype, or undifferentiation marker expression. This evaluation for the reprogramming state is suitable particularly for overviewing whether a large number of cells in a colony mature as iPS cells, and can also be used to confirm that the reprogramming has been completed.

Since alkaline phosphatase is highly expressed in pluripotent stem cells, it is one of markers in an undifferentiated state. The alkaline phosphatase activity can be analyzed, for example, by alkaline phosphatase staining in accordance with a known method. This analysis can be carried out by determining the presence or absence of an alkaline phosphatase activity or by quantitating the alkaline phosphatase activity, through alkaline phosphatase staining. The alkaline phosphatase staining can be carried out, for example, by a process that the cells are formalin-fixed, to which subsequently a substrate solution including a mixture of 5-bromo-4-chloro-3-indolyl phosphate (BCIP) and nitroblue tetrazolium (NBT) is added, and reaction products associated with alkaline phosphatase activity are detected with a microscope.

The karyotype can be analyzed, for example, by analyzing changes in a number and a structure of chromosomes through a G-band analysis.

The undifferentiation marker expression can be analyzed by RT-qPCR of an undifferentiation marker gene, or immunostaining of an undifferentiation marker protein. As the undifferentiation marker, for example, Nanog, Oct3/4, TRA1-60, and TRA1-81 can be used.

### Evaluation for Differentiation Potency

The differentiation potency of the iPS cells can be evaluated, for example, by inducing differentiation into cells in three germ layers, and then analyzing tridermic differentiation marker expression.

The differentiation marker expression can be analyzed by RT-qPCR of a differentiation marker, or immunostaining of a differentiation marker protein. As the differentiation marker, for example, ectoderm markers (PAX6, MAP2), mesoderm markers (α-SMA, Branchyury), and endoderm markers (SOX 17, AFP) can be used. Correspondence between Results of Evaluating Expression State of Transcription Factor and Results of Evaluating Quality of iPS Cells

Correspondence is made between the result of evaluating the expression state of the transcription factor, obtained by the method for analyzing the somatic cell reprogramming described hereinbefore, and the result of evaluating the quality of the iPS cells, obtained by the evaluation for the quality of the iPS cells described hereinbefore. Since the latter evaluation result is based on a known technique, this correspondence can associate the expression state of the transcription factor at the initial stage of reprogramming (e.g., within three weeks after the onset of reprogramming induction) with high-quality iPS cells. Such association can be made, for example, depending on whether or not the expression levels of one or plural transcription factors reach a predetermined threshold value.

Criteria for evaluating the quality of the iPS cells can be set up based on a result of the correspondence between the high-quality iPS cells and the expression state of the transcription factor at the initial stage in the iPS cell reprogramming. Specifically, when a plurality of colonies with a transcription factor exhibiting a specific expression pattern (e.g., an expression pattern exhibiting an expression level of the transcription factor exceeding a predetermined threshold value) can be associated with the high-quality iPS cells identified by a conventional quality evaluation method, this specific expression pattern can be adopted as a quality evaluation criterion for the iPS cells. This makes it possible to carry out a quality evaluation for selecting high-quality iPS cells from the expression state of the transcription factor at the initial stage of reprogramming. Also, this makes it possible to carry out an objective quality evaluation for the iPS cells with a support by numerical data, at an earlier stage than the conventional evaluation based on the colony morphology. In addition to the “evaluation by the method for analyzing the somatic cell reprogramming” described hereinbefore, at least one of the “evaluation for the colony morphology” and the “evaluation for the quality of the iPS cell by confirming expression of the undifferentiation marker and/or differentiation marker” is carried out, so that the high-quality iPS cells can be more accurately selected.

### <<Effect>>

As described hereinbefore, the analysis method according to an embodiment of the disclosed technology allows the somatic cell reprogramming to be quantitatively analyzed with little damage to the cells, at the initial stage of the reprogramming, for example, within three weeks after introduction of the transcription factor into the somatic cells (i.e., the onset of reprogramming induction).

Conventionally, the reprogramming states have been evaluated only by visually observing the colony morphology three to four weeks after the onset of reprogramming induction. As a result of using the analysis method according to the embodiment of the disclosed technology prior to such visual evaluation, the expression state of the transcription factor can be evaluated. In addition, the evaluation for the expression state of the transcription factor becomes possible to grasp the state of the cells at the initial stage of reprogramming (e.g., within three weeks after the onset of reprogramming induction), preferably at the earliest stage of reprogramming (e.g., within one week after the onset of reprogramming induction). Furthermore, based on these evaluation results, an initial screening can be carried out at the initial stage of reprogramming.

In addition, since the analysis method according to an embodiment of the disclosed technology does not use excitation light, the expression intensity of the transcription factor can be quantified in a manner less invasive to the cells than the analysis method with fluorescence. Furthermore, this analysis method is also superior in quantitativity to the analysis method using fluorescence. Specifically, analysis of the expression level using no excitation light but using luminescence is superior to the method using fluorescence from the following viewpoints.

### (i) Low Invasiveness

Irradiation with excitation light is necessary for detecting fluorescent proteins. Irradiation with excitation light is known to bring phototoxicity to cells. For example, the excitation light is involved in generation of reactive oxygen species, causing oxidative damage to DNA due to reactive oxygen species. In addition, membrane blebs (bubbles of membrane) and cell division abnormality occur at an excitation wavelength of a fluorescent protein represented by GFP. Furthermore, the expression itself of the fluorescent protein causes cell death induction.

In contrast, analysis of the expression level using no excitation light but using luminescence has little damage to cells even in a long-term observation. In addition, expression of the luminescent reporter protein is harmless to the cells.

### (ii) High Quantitativity

Factors capable of affecting the quantitativity include presence or absence of autofluorescence, a length of an intracellular half-life of the protein, and a length of time required for protein maturation.

For example, during detection of the fluorescent protein, autofluorescence is often observed.

On the other hand, during detection of the luminescent reporter protein, backgrounds due to autofluorescence are not observed, and thus the analysis method using the luminescent reporter protein is excellent in quantitativity.

In addition, the intracellular half-life of the fluorescent protein is long, for example, substantially 20 hours. For this reason, it is difficult to accurately detect the timing of decrease in the expression level.

On the other hand, the intracellular half-life of the luminescent reporter protein is, for example, substantially three hours. For this reason, the analysis using the luminescent reporter protein is superior in trackability for change of the expression level compared to the analysis using fluorescent protein.

Furthermore, the maturation of the fluorescent protein takes at least three hours and at most 20 hours. Particularly for red fluorescence, the fluorescence matures to a red color through a green fluorescence during the maturation process, and thus, it is difficult to distinguish the red fluorescence from the green fluorescence during the maturation process.

On the other hand, a luminescent reporter protein such as luciferase matures as soon as the expression of the luminescent reporter protein is induced. For this reason, detection using luminescence is suitable for monitoring at the initial stage of reprogramming.

As described hereinbefore, the analysis method according to the embodiment of the disclosed technology is excellent not only in invasiveness but also in quantitativity. Thus, when the analysis method according to the embodiment of the disclosed technology is carried out at the initial stage of reprogramming situated on the most upstream step in the iPS cell preparation process, a slight change in the expression level of the transcription factor can be analyzed with no damage to the cells. In addition, the evaluation for the expression state using luminescence is superior to conventional evaluation methods, i.e., a method based on sensory criteria of an engineer, such as visual evaluation for the colony morphology, from the viewpoint that the evaluation using luminescence is a quantitative and objective method.

In addition, since the analysis method according to an embodiment of the disclosed technology allows coexpression of some extrinsic transcription factors introduced into cells for the purpose of inducing reprogramming with the luminescent reporter protein, the expression level of the transcription factor can be directly analyzed depending on the luminescence intensity. Thereby, the evaluation according to an embodiment of the disclosed technology allows grasp of the actual expression level compared to the conventional indirect evaluation for the expression level using a promoter assay.

The analysis method according to an embodiment of the disclosed technology allows analysis of the expression distribution of the transcription factor in a cell population forming colonies, from a luminescence image, for example. In the art, when establishing the iPS cells, it is common to select the iPS cells in such a way that only a part of the cells in a central portion of a colony is physically separated, from the viewpoint of productivity for the iPS cells. Hence, the expression distribution of the transcription factor in the cell population forming the colony is analyzed, so that cells with appropriately expressed transcription factors can be selected from the cell population in the central portion of the colony without separating the cells, based on image analysis, or an image displayed on displaying means such as a display. From the luminescence intensity and/or the increasing rate of the luminescent quantity on each of the central portion of the colony and the peripheral portions of the colony in a plurality of directions relative to the central portion, the iPS cell establishment process, i.e., the pluripotency acquisition state for each cell can be grasped. Furthermore, a bright field observation is carried out, preferably a bright field image is acquired using a known optical system for phase difference observation, and this bright field image is superposed with the luminescence image, to provide an indicator for evaluating the quality of the iPS cells associated with the morphological change of the cells in the reprogramming process. This observation is preferable particularly from the viewpoint that the evaluation for the quality of the iPS cells, in which the change in the colony thickness is associated with the gene expression level, can be achieved by repeatedly acquiring both the phase difference observation image and the luminescence image in different processes of reprogramming.

Furthermore, the method for setting up the quality evaluation criteria for the iPS cells using the analysis method according to an embodiment of the disclosed technology can make the correspondence between the high-quality iPS cells selected based on the conventional evaluation method and the evaluation result of the expression state of the transcription factor at the initial stage of reprogramming (e.g., within three weeks after the onset of reprogramming induction), preferably at the earliest stage of reprogramming (e.g., within one week after the onset of reprogramming induction), and thus, unprecedented novel quality evaluation criteria for the iPS cells can be set up. In addition, if criteria are set up by this method, cells which are expected to become high-quality iPS cells in the future can be screened in the early stage of reprogramming to increase the establishment efficiency of the high-quality iPS cells.

### EXAMPLES

Hereinafter, Examples of the disclosed technology will be described. In Example, Oct3/4, Sox2, Klf4, L-myc, and Lin28 were used as inducers for reprogramming. Among these inducers, each of Oct3/4, Sox2, and Klf4 is linked to luciferase as a luminescent reporter protein, and the expression state of each inducer was evaluated from the luminescence image.

### 1. Method

### 1-1. Preparation of Vector

In order to induce reprogramming, the following five episomal vectors were prepared.
(1) Vector expressing Oct3/4 and SfRE1 luciferases (pCXLE-hOCT3/4-shp53-SfRE1)
(2) Vector expressing Sox2, Klf4, and OkiMado luciferases (pCXLE-hSK-OkiMado)
(3) Vector expressing L-myc and Lin 28 (pCXLE-hUL)
(4) Vector expressing a mouse p53 dominant negative mutant (pCE-mp53DD)
(5) Vector expressing EBNA1 (pCXB-EBNA1)

These five vectors are referred to as “modified vector set.” A transcription factor was selected based on Okita K, et al., Nat. Methods 2011 May; 8(5): 409-412. A more efficient method to generate integration-free human iPS cells.

Hereinafter, the expression vectors (1) to (5) will be described in detail.

(1) pCXLE-hOCT3/4-shp53-SfRE1

The pCXLE-hOCT3/4-shp53-SfRE1 was prepared such that the SfRE1 luciferase as a luminescent reporter protein was co-expressed with the Oct3/4.

Specifically, the pCXLE-hOCT3/4-shp53 (Addgene) was digested with Kpn I and Bgl II, and an SfRE1 luciferase (SEQ ID NO: 1) was incorporated downstream of the hOCT3/4 through the 2A sequence to prepare the expression vector, as depicted in FIG. 3A.

(2) pCXLE-hSK-OkiMado

The pCXLE-hSK-OkiMado was prepared such that the OkiMado luciferase as a luminescent reporter protein was co-expressed with the Sox2 and Klf4.

Specifically, the pCXLE-hSK (Addgene) was digested with Sph I and Bgl II, and an OkiMado luciferase (SEQ ID NO: 2) was incorporated downstream of KLF4 through the 2A sequence to prepare the expression vector, as depicted in FIG. 3B. In relation to the Kpn I site depicted in FIG. 3B, a sequence was artificially synthesized from the inside of the KLF4 so as to include the Kpn I site and exclude a stop codon, and inserted.

(3) pCXLE-hUL

The pCXLE-hUL was obtained from Addgene.

(4) pCE-mp53DD

The pCE-mp53DD was obtained from Addgene.

(5) pCXB-EBNA1

The pCXB-EBNA1 was obtained from Addgene.

For the induction of reprogramming for comparison, in addition to the vectors (3) to (5), the following vectors (6) and (7) were used. These five vectors are referred to as “control vector set.”

(6) pCXLE-hOCT3/4-shp53

The pCXLE-hOCT3/4-shp53 was obtained from Addgene.

(7) pCXLE-hSK

The pCXLE-hSK was obtained from Addgene.

The base sequences of the SfRE1 luciferase and the OkiMado luciferase are indicated hereinafter.

TABLE 1
(SEQ ID No: 1) SfRE1 luciferase

atggccagcagcatgatgagcaagaaggacctggaagataagaacgtggtg
cacggccccgacccctactacctggtggatgagggcaatgccggccagcag
ctgcacaagaccatcctgagatacgcccagctgcccgacacaatcgccttc
accgacggccacaccaagcgggatgtgacctacgcccactacttcgacctg
acctgcagactggccgagagcctgaagagatacggcctgaacctgcagagc
cggatcgccgtgtgcagcgagaacaacgtggaatttttcatccccgtggtg
gccagcctgtacctgggagtgggagtggcccccaccaacgacatctacaac
gagacagagctgttcaacagcctgaacatcagccagcccaccatcgtgttc
gtgtccaagcgggccctgcacaagatcctggaagtgaagaagcgcatcccc
atcatcaagaccgtggtggtgctggacaccgaagaggacttcatgggctac
cactgcctgcacagctttatgaagcactacctgccccccaacttcgacatc
atgagctacaagcccgaagagttcgcccgggatggacagctggccctgatc
atgaacagcagcggcagcaccggcctgcctaaaggcgtgatgctggcccac
agatccgtggtcgtgcggttcagccactgcaaggaccccgtgttcggcaac
cagatcatccccgacaccgctatcctgaccgtgatccctttccaccacggc
ttcggcatgttcaccaccctgggctacctgacctgtggcttccggatcgtg
ctgctgcggaagttcgacgagcactactttctgaagtgcctgcaggactac
aagatccagtttgccctgctggtgcctaccctgttcagcttcttcgccaag
agcaccctggtggaccagtacgacctgagcaacctgaaagagatcgccagc
ggcggagcccccctggctaaagaagtgggagaggccgtcgccaagcggttt
aagctgcccggcatcagacagggctacggcctgaccgagacaaccagcgcc
gtgatcatcacccccgagggcgaggataagcctggctctacaggcaaggtg
gtgccattcttcagcgccaagatcgtggacctgaacagcggcaagagcgtg
ggccctcaccagaggggagaactctacctgaagggcgacatgatcatgatg
ggctactgcaacaacaaggccgccaccgacgagatgatcgacaaggatggc
tggctgcactggggcgacgttgcctactacgacgaggacggccacttcttc
atcgtggaccggctgaagtccctgatcaagtacaagggctaccaggtggcc
cctgccgaactggaagctgtgctgctgcagcatccctgcatcttcgatgcc
ggcgtgaccggcgtgccagatgatgtggacggcgaactgcctggcgcctgt
gtggtcctggaaaagggcaagcacgtgaccgagcaggaagtgatggactac
gtcgccggccagctgagctgctacaagagactgagaggcggtgtgcgcttc
atcgatgagatccctaagggcctgaccggcaagatcgaccggaaggccctg
aaagaaatcctgaagaaaccccagagcaagatgtga

TABLE 2
(SEQ ID No: 2) OkiMado luciferase

atggaagatgaccacaagaacatcgtgcacggccctgccccattctacccc
ctggaagagggaacagccggcgagcagctgcaccgggccatgaagagatat
gcccaggtgcccggcacaatcgccttcaccgatgcccacgtggaagtgaac
atcacctacagcgagtacttcgagatggcctgccggctggccgagacaatg
aagcgctatggcctgggcctgcagcaccacattgccgtgtgcagcgagaac
agcctgcagttcttcatgcccgtgtgtggcgccctgttcatcggagtggga
gtggcccccaccaacgacatctacaacgagagagagctgtacaacagcctg
agcatcagccagcccaccatcgtgttctgcagcaagcgggccctgcagaaa
atcctgggcgtgcagaaaaagctgcccgtgatcgagaagatcgtgatcctg
gacagccgcgaggactacatgggcaagcagagcatgtacagcttcatcgag
agccatctgcccgctggcttcaacgagtacgactacgtgcccgacaccttc
gacagagagacagccaccgccctgatcatgaacagcagcggctctaccggc
ctgcccaagggcgtggaactgacccacaagaatgtgtgcgtgcggttcagc
cactgccgggaccctgtgttcggcaaccagatcatccccgacaccgctatc
ctgaccgtgatccccttccaccacggcttcggcatgttcaccaccctgggc
tacctgacctgcggcttccggatcgtgctgatgtacagattcgaggaagaa
ctgttcctgcggagcctgcaggactacaagatccagagcgccctgctggtg
cctaccctgttcagcttcttcgccaagagcaccctggtggataagtacgac
ctgagcaacctgcacgagatcgcctctggcggagcccccctggctaaagaa
gtgggagaggccgtggccaagcggttcaagctgcctggcatcagacagggc
tacggcctgaccgagacaacctctgccgtgatcatcacccccaggggcgac
gataagcctggcgcctgtggaaaggtggccccatttttcagcgccaagatt
gtggacctggacaccagcaagacactgggcgtgaaccagaggggcgagctg
tgtctgaagggccccatgattatgaagggctacgtgaacaaccccgaggcc
accaatgccctgatcgacaaggatggctggctgcactctggcgacctggcc
tactacgacaaggacggccacttcttcatcgtggaccggctgaagtccctg
atcaagtaccagggctaccaggtgccacccgccgagctggaatctatcctg
ctgcagcatcccttcatcttcgatgccggggtggccggcatccctgatgct
gatgctggcgaactgcctgccgctgtggtggtgctggaagagggcaagacc
atgaccgagcaggaagtgatggactacgtggccggacaagtgaccgccagc
aagaggctgagaggcggcgtgaagttcgtggacgaggtgccaaagggcctg
acaggcaagatcgacagccggaagatccgcgagatgctgacaatgggcaag
aaaagcaagctgtga

### 1-2. Gene Introduction and Cell Culture

A human peripheral blood mononuclear cell (PBMC; obtained from Cellular Technology Limited) was thawed and cultured in a medium (AK02, Ajinomoto Co., Inc.) supplemented with IL3, IL6, SCF, TPO, Flt3-Ligand, and CSF. For the cell culture, a 24-well culture plate was used. Cells were seeded at a density of 2.5×10⁶ cells/well and cultured at 37° C. in a 5% CO₂ environment for seven days without exchanging the medium.

After the 7-day culture, the “modified vector set” was introduced into the PBMC by electroporation using Amaxa (Lonza). As a control, the “control vector set” was introduced to the PBMC in the same manner.

Subsequently, the PBMC transfected with the vector was seeded on a 6-well culture plate coated with a coating agent (iMatrix, Nippi, Inc.) in advance and cultured in a medium (AK02, Ajinomoto Co., Inc.) supplemented with IL3, IL6, SCF, TPO, Flt3-Ligand and CSF at 37° C. in a 5% CO₂ environment overnight. The seeding density was 2×10⁶ cells/well.

On and after the next day, i.e., on and after the second day following the vector introduction, the culture was continued while adding 1.5 mL of medium (AK02, Ajinomoto Co., Inc.) every day. On the eighth day after the vector introduction, the medium after formation of the iPS cell-like colony was exchanged with 2 mL of medium (AK02, Ajinomoto Co., Inc.), and then cultured.

### 1-3. Acquisition of Luminescence Image

The “modified vector set” was introduced into cells, and luminescence images were acquired from the next day to the eighth day when the iPS cells are expected to begin to form.

Luminescence attributed to the catalytic action of the luciferase was observed by adding luciferin at a final concentration of 1 mM to the medium.

For acquiring the luminescence images, the luminescent microscope LV200 (Olympus Corporation) was used, and for a CCD camera, ImagEM (Hamamatsu Photonics K. K.) was used. As an imaging condition, a binning was set to 1×1, and an EM-gain was set to 1200. The cell luminescence on the first day after the vector introduction was imaged using a 20-power objective lens with a camera exposure time of three minutes to acquire luminescence images. The luminescence of colony on the eighth day was imaged using a 4- or 20-power objective lens with a camera exposure time of five minutes to acquire luminescence images.

Luminescence attributed to expression of the SfRE1 luciferase was spectrally separated using 515-560 HQ filter. Luminescence attributed to expression of the OkiMado luciferase was spectrally separated using 610 ALP filter.

### (1-4. Quantification of Luminescence Intensity)

On each of the luminescence images taken by imaging cells on the first day after the vector introduction and an iPS-like cell colony on the eighth day after the vector introduction, a luminescence intensity at a wavelength range of luminescence attributed to the SfRE1 luciferase and a luminescence intensity at a wavelength range of luminescence attributed to the OkiMado luciferase were quantified. For quantification, an image analysis software (Cell Sens (Olympus Corporation)) was used. At this time, a portion where the wavelength ranges of the SfRE1 luciferase and the OkiMado luciferase overlap with each other was excluded by unmixing treatment.

Prior to the unmixing treatment, the PBMCs individually transfected with each of the OkiMado luciferase and the SfRE1 luciferase were subjected to luminescence imaging to calculate a filter transmittance of each luciferase. For the luminescent component OkiMado, filter transmittances “T1o” and “T2o” were determined from the luminance values of the luminescence imaging results using the 515-560 HQ filter and the 610ALP filter relative to the luminance values of the luminescence imaging results without using the filter. Also for the luminescent component SfRE1, the filter transmittances “T1s” and “T2s” were determined in the same manner. Signal values measured using the 515-560 HQ filter and the 610ALP filter were defined as “F1” and “F2.” Each filter measurement value is expressed as a sum of a product obtained by multiplying each luminescent component by the ratio (transmittance) measured using the filter. From this, the following simultaneous equations:

F1=T1o·OkiMado+T1s·SfRE1

F2=T2o·OkiMado+T2s·SfRE1

can be solved to obtain each luminescent component of the OkiMado and the SfRE1.

### (1-5. Evaluation for Alkaline Phosphatase Activity)

iPS-like cell colonies obtained on the eighth day after introduction of the “modified vector set” and confirmed to have luminescence, and iPS-like cell colonies obtained on the eighth day after introduction of the “control vector set” were fixed with formalin, and then, treated by alkaline phosphatase staining using a commercially available kit.

### 2. Results and Discussion

(2-1. Acquisition of Cell Luminescence Image on the First Day after Introduction of Vector)

Luminescence on the PBMC on the day after introduction of the “modified vector set” was observed to acquire the luminescence images in FIG. 4.

The middle image in FIG. 4 depicts a luminescence image attributed to the SfRE1 luciferase, which is spectrally separated with the 515-560 HQ filter.

The lower image in FIG. 4 depicts a luminescence image attributed to the OkiMado luciferase, which is spectrally separated with the 610 ALP filter.

The upper image in FIG. 4 depicts an image obtained by superposing the middle image, the lower image, and the phase difference observation image in FIG. 4.

As depicted in FIG. 4, from the first day after introduction of the vector, luminescence attributed to the SfRE1 luciferase exhibiting Oct3/4 expression, and luminescence attributed to the OkiMado luciferase exhibiting Sox2 and Klf4 expressions were confirmed. As is obvious from these images, luminescence was observed only on some cells presumed to be transfected with the vectors.

These results indicated that any luciferases having different wavelength ranges could be observed by spectrally separating the luminescences attributed to a plurality of luciferases in the same cell, and that the expression of the transcription factor could be confirmed from the luminescence image by using vectors configured to co-express the luminescent reporter protein with the transcription factor.

In addition, it was indicated that, since the positions of cells expressing the Oct3/4, Sox2, and Klf4 could be visually observed in the whole seeded PBMCs, the same cell could be pursued also in the subsequent time-dependent analysis by recording the cell position information.

(2-2. Acquisition of iPS-like Cell Colony Luminescence Image on the eighth Day after Introduction of Vector)

Luminescence on the iPS-like cell colony on the eighth day after introduction of the “modified vector set” was observed to acquire the luminescence images depicted in FIG. 5 and FIG. 6.

FIG. 5 depicts an image taken by a 4-power objective lens.

The upper image in FIG. 5 depicts a phase difference observation image (bright field).

The middle image in FIG. 5 depicts a luminescence image attributed to the SfRE1 luciferase, which is spectrally separated with the 515-560 HQ filter.

The lower image in FIG. 5 depicts a luminescence image attributed to the OkiMado luciferase, which is spectrally separated with the 610 ALP filter.

Colonization could be observed as depicted in the phase difference observation image in the upper image in FIG. 5. This indicates that colonization can be evaluated by acquiring a phase difference observation image.

As depicted in the luminescence image in the middle image in FIG. 5, luminescence attributed to the SfRE1 luciferase was widely observed in the colonies. This indicates that the Oct3/4 is expressed in many colonizing cells.

Luminescence attributed to the OkiMado luciferase was widely observed in colonies, as depicted in the luminescence image in the lower image of FIG. 5. This indicates that the Sox2 and Klf4 are expressed in many colonizing cells.

FIG. 6 depicts a result of imaging the ranges indicated by white squares in the upper image in FIG. 5 with a 20-power objective lens. FIG. 6 depicts an image obtained by superposing the luminescence image attributed to the SfRE1 luciferase which is spectrally separated by the 515-560 HQ filter, the luminescence image attributed to the OkiMado luciferase which is spectrally separated by the 610 ALP filter, and the phase difference observation image.

The results in FIG. 5 and FIG. 6 indicated that the cells normally transfected with the vectors proliferated and colonized. In addition, the luminescence image could be obtained even eight days after the vector introduction, indicating that the expression of the SfRE1 luciferase and the OkiMado luciferase accompanying the expression of the transcription factor was not transient but persisted over a long period also after colonization. These results suggested that the luminescence images could be acquired over time.

Furthermore, each part of the colony could be observed, revealing that the expression state of the transcription factor at each site in the colony could be observed, and homogeneity of the expression level of each transcription factor in the colony could be visually confirmed. This suggests that, whereas the conventional analysis for the expression level of the transcription factor by RT-qPCR has been possible only for the whole colony, the method according to the disclosed technology allows more precise analysis.

### 2-3. Quantification of Luminescence Intensity

The luminescence intensity of luminescence attributed to the SfRE1 luciferase and the luminescence intensity of luminescence attributed to the OkiMado luciferase were quantified from the luminescence images.

Areas a, b, c, d, and e depicted in the upper image in FIG. 4 were arbitrarily selected from the luminescence image so as to include cells with observable luminescences attributed to the SfRE1 luciferase and the OkiMado luciferase. For these areas, the luminescence intensity at the wavelength range of luminescence attributed to the SfRE1 luciferase and the luminescence intensity at the wavelength range of luminescence attributed to the OkiMado luciferase were respectively quantified and graphed, and the results are indicated in FIG. 7.

For the cells included in the areas a, b, c, d, and e, the luminescence intensity of luminescence attributed to the SfRE1 luciferase co-expressed with the Oct3/4, and the luminescence intensity of luminescence attributed to the OkiMado luciferase co-expressed with the Sox2 and the Klf4 were independently quantified, and the results are indicated in the graph of FIG. 7.

The results revealed that the luminescence intensity of luminescence attributed to the expression of luciferase accompanying the expression of the transcription factor could be quantified for each cell.

Areas f, g, h, i, and j depicted in FIG. 6 were arbitrarily selected as sites with observable luminescences attributed to the SfRE1 luciferase and the OkiMado luciferase, from the luminescence image of the iPS-like cell colony. For these areas, the luminescence intensities at the wavelength range of luminescence attributed to the SfRE1 luciferase and at the wavelength range of luminescence attributed to the OkiMado luciferase were respectively quantified and graphed, and the results are indicated in FIG. 8.

For the areas f, g, h, i, and j, the luminescence intensity of luminescence attributed to the SfRE1 luciferase co-expressed with the Oct3/4, and the luminescence intensity of luminescence attributed to the OkiMado luciferase co-expressed with the Sox2 and the Klf4 were independently quantified, and the results are indicated in the graph of FIG. 8.

These results revealed that the luminescence intensity of luminescence attributed to the expression of luciferase accompanying the expression of the transcription factor could be quantified also for the colonizing cells. In addition, the results of FIG. 7 and FIG. 8 revealed that the gene expression levels related to all transcription factors were increased on the first day and the eighth day after the vector introduction and could be quantified, and thus, the step of quantifying the luminescence intensity could be repeated over time.

Furthermore, it was indicated that a ratio of luminescence intensities of luminescence attributed to expressions of a plurality of luciferases could be determined, because the luminescence intensities were acquired as numerical data.

Furthermore, since the expression of luciferase accompanies the expression of the transcription factor linked to the upstream of the luciferase via the 2A sequence, the quantified luminescence intensity reflects the expression level of the transcription factor. For this reason, it was also suggested that the expression level of the transcription factor could be calculated from the quantification of the luminescence intensity by previously determining the relationship between the luminescence intensity of the luciferase and the expression level of the transcription factor quantifiable with a technique such as RT-qPCR in advance. Thus, it was indicated that the expression state of the transcription factor could be evaluated based on the luminescence image.

### 2-4. Evaluation for Reprogramming State

For the PBMC transfected with the “modified vector set” and the PBMC transfected with the “control vector set,” the reprogramming states on the eighth day after the transfection were evaluated by evaluating an alkaline phosphatase activity as a pluripotency marker.

FIG. 9 depicts a bright field image in which an alkaline phosphatase activity of the PBMC on the eighth day after introduction of the modified vector set was detected.

FIG. 10 depicts a bright field image in which an alkaline phosphatase activity of the PBMC on the eighth day after introduction of the control vector set was detected.

As depicted in FIG. 9, the iPS-like cell colony obtained by introducing the modified vector was stained, and proved positive in the alkaline phosphatase activity.

This result was equivalent to the result of staining the iPS-like cell colony obtained by introducing the control vector set depicted in FIG. 10.

These results suggested that also when the iPS cells were induced by introducing the modified vector set, the reprogramming smoothly proceeded equivalently to a case in which the iPS cells were induced by introducing the control vector set. This indicates that the reprogramming of somatic cells can be evaluated even by using a vector modified so as to co-express the luciferase with the transcription factor. It is expected that this fact also applies to a case of changing the combination of the transcription factor used for induction of the iPS cells with the luciferase.

Furthermore, in FIG. 5 and FIG. 6, luminescence was observed in many cells forming the iPS-like cell colony, and the iPS-like cell colony was positive in alkaline phosphatase activity, indicating relationship of the luminescence with the reprogramming state. In addition, as depicted in FIG. 6, by using the phase difference image in combination, it could be confirmed whether or not the colony had grown to the iPS-like cell colony, without using a reagent. In particular, when superposing the phase difference image and the luminescence image, it was possible to compare the luminescence intensities between the sites having different thicknesses in the colony, suggesting that using the phase difference image in combination improved the accuracy of the comprehensive determination. The method according to the disclosed technology allows the expression of the transcription factor in the reprogramming process to be observed and quantified from the luminescence image. Furthermore, the results were made to correspond to the results of evaluating the quality of the iPS cells, and accordingly, it was suggested that criteria for the expression state of the transcription factor exhibited by the high-quality iPS cells in the initialization process could be set up. In addition, if the quality evaluation criteria for the iPS cells are set up in this way, it was suggested that the high-quality iPS cell colony could be selected using a quantitative method minimally invasive to cells at the initial stage of reprogramming. Furthermore, the method according to the disclosed technology can also be used as an evaluation method for searching a transcription factor optimal for growth of the iPS cells.

While various embodiments of the disclosed technology have been described above, it should be understood that they have been presented by way of example only, and not of limitation. Likewise, the various diagrams may depict an example schematic or other configuration for the disclosed technology, which is done to aid in understanding the features and functionality that can be included in the disclosed technology. The disclosed technology is not restricted to the illustrated example schematic or configurations, but the desired features can be implemented using a variety of alternative illustrations and configurations. Indeed, it will be apparent to one of skill in the art how alternative functional, logical or physical locations and configurations can be implemented to implement the desired features of the technology disclosed herein.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one”, “one or more” or the like; and adjectives such as “conventional”, “traditional”, “normal”, “standard”, “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more”, “at least”, “but not limited to” or other like phrases in some instances shall not be read to mean that the narrower case is intended or required in instances where such broadening phrases may be absent. Additionally, the various embodiments set forth herein are described in terms of exemplary schematics, block diagrams, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular configuration.

What is claimed is:

1. A method for analyzing a somatic cell reprogramming, the method comprising:
acquiring luminescence images associated with luminescences attributed to expression of luminescent reporter proteins during a period of culturing cells transfected with a first nucleic acid and a second nucleic acid, the first nucleic acid encoding a plurality types of transcription factors required for the somatic cell reprogramming, the second nucleic acid encoding the luminescent reporter proteins being configured to be co-expressed with at least one of the plurality types of transcription factors;
quantifying luminescence intensities of the luminescences based on the luminescence images; and
evaluating expression states of the plurality types of transcription factors to be co-expressed with the luminescent reporter proteins based on the luminescence intensities, wherein the expression states are indicators for evaluating whether a pluripotency has been acquired in the cell reprogramming process.

2. The method of claim 1, wherein the evaluating the expression states of the transcription factors includes determining expression levels of the plurality types of transcription factors from a result of quantifying the luminescence intensities.

3. The method of claim 1, wherein the acquiring luminescence images is performed at least once within three weeks after introducing the first nucleic acid to the cells.

4. The method of claim 1, wherein the acquiring luminescence images is performed at least once within one week after introduction the first nucleic acid into the cells.

5. The method of claim 1, wherein
the acquiring luminescence images is repeatedly performed over time, and
the evaluating expression states includes evaluating the time-dependent change in the expression states of the plurality types of transcription factors to be co-expressed with the luminescent reporter proteins.

6. The method of claim 1, wherein the plurality types of transcription factors to be co-expressed with the luminescent reporter proteins are at least one selected from a group including Oct3/4, Klf4, Sox2, c-myc, Lin 28, and L-myc.

7. The method of claim 1, wherein
the plurality types of transcription factors include two or more types of transcription factors so as to co-expressed with the luminescent reporter proteins,
each of the luminescent reporter proteins to be co-expressed with two or more types of the transcription factors is a luminescent reporter protein having a luminescence property allowing the luminescent reporter protein to be detected distinguishably with any other luminescent reporter proteins,
the luminescence images are images in which information of the luminescence attributed to each expression of the luminescent reporter proteins configured to be distinguished from information of luminescences attributed to expression of any other luminescent reporter proteins,
the quantifying luminescence intensities includes individually quantifying the luminescence intensity of luminescence attributed to each expression of the luminescent reporter proteins based on the luminescence images.

8. The method of claim 7, wherein the evaluating expression states includes determining the expression levels of the plurality types of transcription factors from the individually quantified luminescence intensities.

9. The method of claim 7, wherein the evaluating expression states includes determining a ratio of the luminescence intensities from the individually quantified luminescence intensities.

10. The method of claim 1, further comprising:
acquiring bright field images of the cells to evaluate colonization of the cells.

11. The method of claim 1, wherein the luminescent reporter proteins are bioluminescent reporter proteins.

12. The method of claim 11, wherein the luminescent reporter proteins are luciferase.

13. The method of claim 1, wherein the acquiring luminescence images is performed in a light-shielded environment.

14. The method of claim 1, wherein the first nucleic acid and the second nucleic acid are introduced into the cells in such a way that the nucleic acids are in a form of episomal vectors.

15. A method for setting up quality evaluation criteria for iPS cells, the setting-up method comprising:
applying a method for analyzing a somatic cell reprogramming in which the method for analyzing a somatic cell reprogramming includes
acquiring luminescence images associated with luminescences attributed to expression of luminescent reporter proteins during a period of culturing cells transfected with a first nucleic acid and a second nucleic acid, the first nucleic acid encoding a plurality types of transcription factors required for the somatic cell reprogramming, the second nucleic acid encoding the luminescent reporter proteins being configured to be co-expressed with at least one of the plurality types of transcription factors;
quantifying luminescence intensities of the luminescence based on the luminescence images; and
evaluating expression states of the plurality types of transcription factors to be co-expressed with the luminescent reporter proteins based on the luminescence intensities, wherein the expression states are indicators for evaluating whether a pluripotency has been acquired in the cell reprogramming process
obtaining first results of evaluating the expression states of the transcription factors;
evaluating qualities of iPS cells prepared by reprogramming the cells to obtain second results of evaluating the qualities of the iPS cells; and
setting up the quality evaluation criteria for the iPS cells based on the relationship between the first results of evaluating the expression states of the transcription factors and the second results of evaluating the qualities of the iPS cells.

16. The method of claim 15, wherein the evaluating qualities of the iPS cells includes evaluating a colony morphology formed by the cells.

17. The method of claim 15, wherein the evaluating qualities of the iPS cells includes evaluating the reprogramming states of the cells by analyzing an alkaline phosphatase activity, a karyotype, or undifferentiation marker expression.

18. The method of claim 15, wherein the evaluating qualities of the iPS cells includes evaluating a differentiation potency of the iPS cells by inducing differentiation into three germ layers, and then analyzing differentiation marker expression.


SEQUENCE LISTING (2 sequences)

SEQ ID NO: 1 - DNA, 1668 bp, Artificial Sequence
  SfRE1 luciferase
atggccagcagcatgatgagcaagaaggacctggaagataagaacgtggtgcacggccccgacccctactacctggtggatgagggcaatgccggccagcagctgcacaagaccatcctgagatacgcccagctgcccgacacaatcgccttcaccgacggccacaccaagcgggatgtgacctacgcccactacttcgacctgacctgcagactggccgagagcctgaagagatacggcctgaacctgcagagccggatcgccgtgtgcagcgagaacaacgtggaatttttcatccccgtggtggccagcctgtacctgggagtgggagtggcccccaccaacgacatctacaacgagacagagctgttcaacagcctgaacatcagccagcccaccatcgtgttcgtgtccaagcgggccctgcacaagatcctggaagtgaagaagcgcatccccatcatcaagaccgtggtggtgctggacaccgaagaggacttcatgggctaccactgcctgcacagctttatgaagcactacctgccccccaacttcgacatcatgagctacaagcccgaagagttcgcccgggatggacagctggccctgatcatgaacagcagcggcagcaccggcctgcctaaaggcgtgatgctggcccacagatccgtggtcgtgcggttcagccactgcaaggaccccgtgttcggcaaccagatcatccccgacaccgctatcctgaccgtgatccctttccaccacggcttcggcatgttcaccaccctgggctacctgacctgtggcttccggatcgtgctgctgcggaagttcgacgagcactactttctgaagtgcctgcaggactacaagatccagtttgccctgctggtgcctaccctgttcagcttcttcgccaagagcaccctggtggaccagtacgacctgagcaacctgaaagagatcgccagcggcggagcccccctggctaaagaagtgggagaggccgtcgccaagcggtttaagctgcccggcatcagacagggctacggcctgaccgagacaaccagcgccgtgatcatcacccccgagggcgaggataagcctggctctacaggcaaggtggtgccattcttcagcgccaagatcgtggacctgaacagcggcaagagcgtgggccctcaccagaggggagaactctacctgaagggcgacatgatcatgatgggctactgcaacaacaaggccgccaccgacgagatgatcgacaaggatggctggctgcactccggcgacgttgcctactacgacgaggacggccacttcttcatcgtggaccggctgaagtccctgatcaagtacaagggctaccaggtggcccctgccgaactggaagctgtgctgctgcagcatccctgcatcttcgatgccggcgtgaccggcgtgccagatgatgtggacggcgaactgcctggcgcctgtgtggtcctggaaaagggcaagcacgtgaccgagcaggaagtgatggactacgtcgccggccagctgagctgctacaagagactgagaggcggtgtgcgcttcatcgatgagatccctaagggcctgaccggcaagatcgaccggaaggccctgaaagaaatcctgaagaaaccccagagcaagatgtga

SEQ ID NO: 2 - DNA, 1647 bp, Artificial Sequence
  OkiMado luciferase
atggaagatgaccacaagaacatcgtgcacggccctgccccattctaccccctggaagagggaacagccggcgagcagctgcaccgggccatgaagagatatgcccaggtgcccggcacaatcgccttcaccgatgcccacgtggaagtgaacatcacctacagcgagtacttcgagatggcctgccggctggccgagacaatgaagcgctatggcctgggcctgcagcaccacattgccgtgtgcagcgagaacagcctgcagttcttcatgcccgtgtgtggcgccctgttcatcggagtgggagtggcccccaccaacgacatctacaacgagagagagctgtacaacagcctgagcatcagccagcccaccatcgtgttctgcagcaagcgggccctgcagaaaatcctgggcgtgcagaaaaagctgcccgtgatcgagaagatcgtgatcctggacagccgcgaggactacatgggcaagcagagcatgtacagcttcatcgagagccatctgcccgctggcttcaacgagtacgactacgtgcccgacaccttcgacagagagacagccaccgccctgatcatgaacagcagcggctctaccggcctgcccaagggcgtggaactgacccacaagaatgtgtgcgtgcggttcagccactgccgggaccctgtgttcggcaaccagatcatccccgacaccgctatcctgaccgtgatccccttccaccacggcttcggcatgttcaccaccctgggctacctgacctgcggcttccggatcgtgctgatgtacagattcgaggaagaactgttcctgcggagcctgcaggactacaagatccagagcgccctgctggtgcctaccctgttcagcttcttcgccaagagcaccctggtggataagtacgacctgagcaacctgcacgagatcgcctctggcggagcccccctggctaaagaagtgggagaggccgtggccaagcggttcaagctgcctggcatcagacagggctacggcctgaccgagacaacctctgccgtgatcatcacccccaggggcgacgataagcctggcgcctgtggaaaggtggccccatttttcagcgccaagattgtggacctggacaccagcaagacactgggcgtgaaccagaggggcgagctgtgtctgaagggccccatgattatgaagggctacgtgaacaaccccgaggccaccaatgccctgatcgacaaggatggctggctgcactctggcgacctggcctactacgacaaggacggccacttcttcatcgtggaccggctgaagtccctgatcaagtaccagggctaccaggtgccacccgccgagctggaatctatcctgctgcagcatcccttcatcttcgatgccggggtggccggcatccctgatgctgatgctggcgaactgcctgccgctgtggtggtgctggaagagggcaagaccatgaccgagcaggaagtgatggactacgtggccggacaagtgaccgccagcaagaggctgagaggcggcgtgaagttcgtggacgaggtgccaaagggcctgacaggcaagatcgacagccggaagatccgcgagatgctgacaatgggcaagaaaagcaagctgtga